(19) United States
(12) Patent Application Publication
(10) Pub. No.: US 2008/0103371 A1
(43) Pub. Date: May 1, 2008

(54) INTERACTIVE METHOD FOR FACILITATING PATIENT COMPLIANCE DURING A HEALTHCARE PROTOCOL

(75) Inventors: James Rosenblum, Chicago, IL (US); Mark Mulert, Deerfield, IL (US); Chris Weiland, Evanston, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: EMMI Solutions, LLC, Chicago, IL (US)

(21) Appl. No.: 11/927,922

(22) Filed: Oct. 30, 2007

Related U.S. Application Data
(60) Provisional application No. 60/863,463, filed on Oct. 30, 2006.

(51) Int. Cl. A61B 5/00

(52) U.S. Cl. 600/300

(57) ABSTRACT

A health care insurance plan, healthcare professional, or other entity may recognize that a patient or member is not in compliance with a treatment protocol, best practices, or guidelines. Entities may desire to communicate this fact along with an action needed to be performed to comply with the protocol, and, thereby, facilitate completion of the action. An interactive system and method may facilitate patient compliance during a healthcare protocol by providing the patient information to make an informed decision regarding the protocol. Further, the system and method may allow a patient to directly implement a portion of the protocol to bring him or her back into compliance with the protocol. An inexpensive, personalized, multimedia interaction specifically designed to motivate a patient and facilitate an action which closes a “Gap in Care,” brings the patient into compliance, and tracks the interaction.

### RELATED APPLICATIONS

This application claims priority from U.S. Patent Application Ser. No. 60/863,463, which was filed on Oct. 30, 2006, entitled “Interactive Method for Facilitating Patient Compliance During a Course of Treatment” the entire contents of which are expressly incorporated by reference herein.

### FIELD OF THE INVENTION

This patent relates to the field of digital information distribution, and more particularly, to interactive methods for delivering information to a healthcare consumer which motivates the healthcare consumer to take action and facilitates such action which will improve a healthcare consumer's compliance with desired best practice health care protocol.

### BACKGROUND

Health care providers strive to provide timely and accurate information to their patients. Interactive multi-media presentations provide an effective means for presenting health care information. Further, information dissemination from the health care provider as well as patient feedback may be facilitated when coupled with computer network systems. For example, patients undergoing surgical procedures may receive information through a Web-based informed consent process. One example of an automated system for completing the informed consent process is disclosed by U.S. patent application Ser. No. 10/410,749, entitled “Enhanced System and Method for Enhancing and Supplementing the Informed Consent Process of a Patient Undergoing a Medical Procedure,” the entire contents of which is hereby expressly incorporated by reference herein.

However, patients living with protracted diseases or medical devices may be restricted in their ability to receive accurate information from their health care provider. In a typical scenario, a patient receives information during a live, in-person appointment or, alternatively, a phone conversation. The doctor and patient may discuss symptoms, test results, medical device use and maintenance, or other health care subjects during a consultation. Courses of treatment for patients with protracted diseases or patients with long-term medical devices, such as pacemakers, may also require follow-up appointments to check their progress, to modify the course of treatment, or to conduct patient testing. Some patients may decide to defer implementing doctor recommendations such as making future appointments for consultation or testing, or may forget to follow their doctor's guidance. Once a patient leaves the provider's office or completes a phone call, the provider must rely on the patient's own interest in maintaining his or her health to complete instructions, schedule testing, or otherwise perform recommended actions. Further, testing that is completed to investigate the progress of a particular symptom or disease may reveal other health care concerns that were not the focus of the original consultation or test. Thus, providing fast and accurate health care information to the patient while considering multiple aspects of the patient's current diagnosis, medical device, or surgical procedure, combined with simple and encouraging measures for patient participation in the course of treatment may be desired.

A health care insurance plan, healthcare professional, or other entity may recognize that a patient or member is not in compliance with a treatment protocol, best practices, or guidelines (i.e., identification of a “Gap in Care”). These entities may desire to effectively communicate this fact along with an action needed to be performed to comply with the treatment protocol, and, thereby, facilitate completion of the action. Conventional methodologies, such as telephone calls, face-to-face visits, interventions, or mail reminders are either cost prohibitive or ineffective. Therefore, an inexpensive, personalized, and short multimedia interaction specifically designed to motivate a patient, health plan member, or healthcare consumer to close the Gap in Care, facilitate the action which closes the Gap in Care, and track the interaction may be highly beneficial.

### BRIEF DESCRIPTION OF THE DRAWINGS

FIG. 1 is an exemplary illustration of a computer network;

FIG. 2 is an illustration of a computing device;

FIG. 3 is a block diagram of a method for facilitating a patient course of treatment;

FIGS. 4a through 4l illustrate a patient's view of the method for facilitating a patient course of treatment.

### DETAILED DESCRIPTION OF THE INVENTION

Although the following text sets forth a detailed description of numerous different embodiments, it should be understood that the legal scope of the invention is defined by the words of the claims set forth at the end of this patent. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

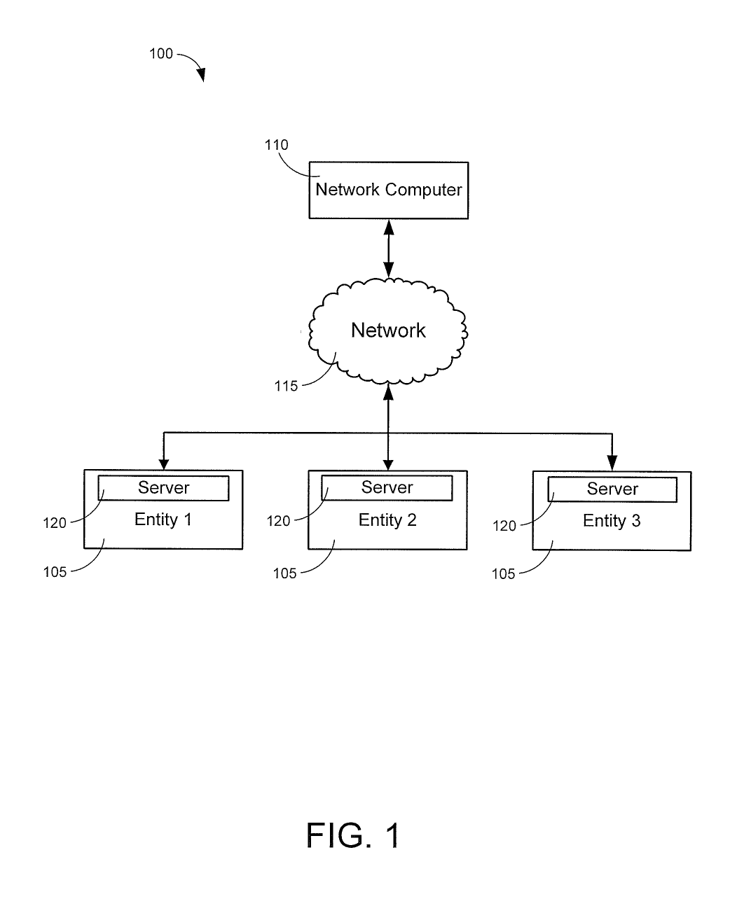

FIG. 1 illustrates an embodiment of a data network 100 including a first group of facilities or entities 105 operatively coupled to a network computer 110 via a network 115. The entities 105 may be physically co-located or geographically disparate. The plurality of entities 105 may be located, by way of example rather than limitation, in separate geographic locations from each other, in different areas of the same city, or in different states. Generally, the entities 105 may represent any of the different types of entities that may be involved in a patient's health care. For example, the entities 105 may represent patients, healthcare providers or professionals (e.g., registered nurses, doctors, therapists, etc.), health insurance providers or administrators, benefits counselors, employee health benefits plans, employer on-site health clinics, and compliance managers. Any of the entities 105 may also be an intermediary between a patient and any of the other entities 105 described above.

The network 115 may be provided using a wide variety of techniques that are well known to those skilled in the art for the transfer of electronic data. For example, the network 115 may comprise dedicated access lines, plain ordinary telephone lines, satellite links, combinations of these, etc. Additionally, the network 115 may include a plurality of network computers or server computers (not shown), each of which may be operatively interconnected in a known manner. Where the network 115 comprises the Internet, data communication may take place over the network 115 via an Internet communication protocol.

The network computer 110 may be a personal computer or a server computer of the type commonly employed in networking solutions. The network computer 110 may be used by an entity 105 to accumulate, analyze, and download health care provider and patient data, or may be used to direct a patient to complete an action that may bring him or her into compliance with a treatment plan or other protocol. For example, the network computer 110 may periodically receive data from each of the entities 105 indicative of information pertaining to a patient health record, provider recommended course of action, treatment plan or other protocol, test results, historic test results, compliance information, etc. A patient may use the network computer 110 to access and view information served from other network computers or servers 120 at the entities 105. For example, as a client/server model, the entities 105 may include one or more servers 120 that may be utilized to store any of the information described herein and to serve the information to a network computer 110 acting as the client.

In one embodiment, the network computer 110 or any of the entities 105 includes an interface to a health records management system at a healthcare facility. For example, the network computer 110 may be connected to a MyChart® electronic health record (EHR) system produced by the Epic Systems Corporation of Verona, Wisconson, or any other type of distributed system that may be used to facilitate a patient's compliance with a healthcare protocol. From a network computer 110, a patient may log into an EHR system that is communicatively coupled to a server 120 within an entity 105.

Although the data network 100 is shown to include one network computer 110 and three entities 105, it should be understood that different numbers of computers and entities may be utilized. For example, the network 100 may include a plurality of network computers 110 and dozens of entities 105, all of which may be interconnected via the network 115. According to the disclosed example, this configuration may provide several advantages, such as, for example, enabling nearly real time uploads and downloads of information as well as periodic uploads and downloads of information. This provides for a primary backup of all the information generated in the process of updating and accumulating provider and patient data.

The computer 110 may be connected to a network, including local area networks (LANs), wide area networks (WANs), portions of the Internet such as a private Internet, a secure Internet, a value-added network, or a virtual private network. Suitable network computer 110 may also include personal computers, laptops, workstations, disconnectable mobile computers, mainframes, information appliances, personal digital assistants, and other handheld and/or embedded processing systems. The signal lines that support communications links to a computer 110 may include twisted pair, coaxial, or optical fiber cables, telephone lines, satellites, microwave relays, modulated AC power lines, and other data transmission “wires” known to those of skill in the art. Further, signals may be transferred wirelessly through a wireless network or wireless LAN (WLAN) using any suitable wireless transmission protocol, such as the IEEE series of 802.x standards. Although particular individual and network computer systems and components are shown, those of skill in the art will appreciate that the present invention also works with a variety of other networks and computers.

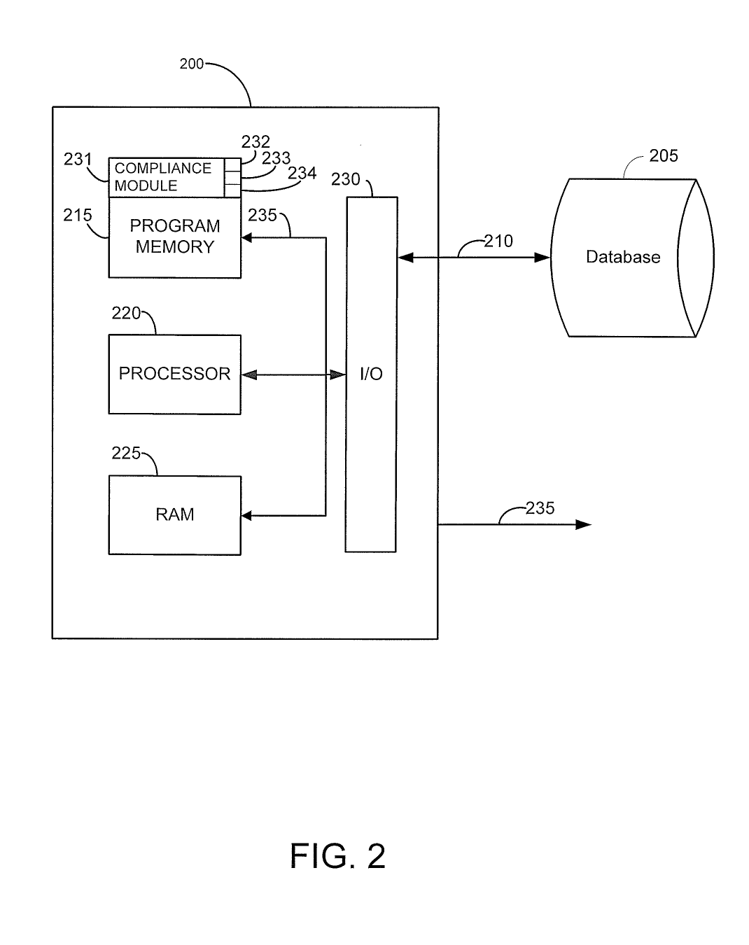

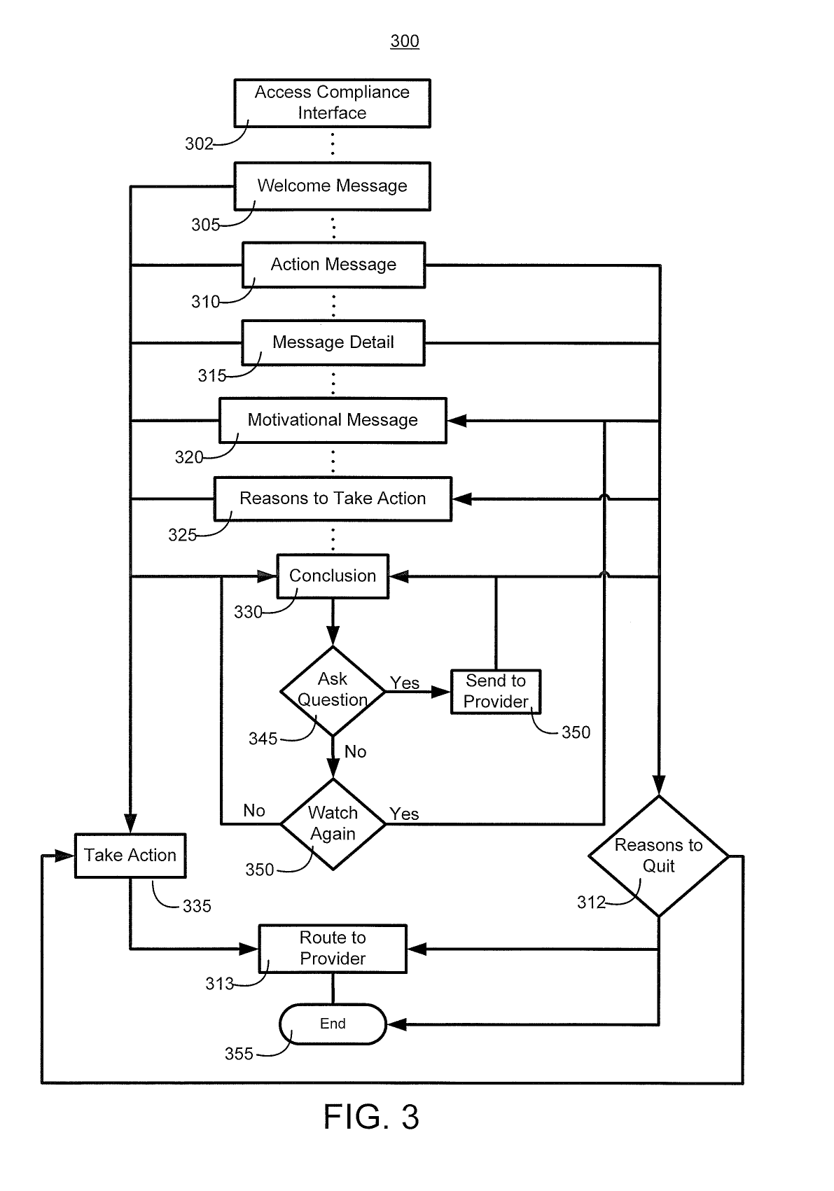

FIG. 2 is a schematic diagram of one possible embodiment of the network computer 110 shown in FIG. 1. The network computer 110 may have a controller 200 that is operatively connected to a database 205 via a link 210. It should be noted that, while not shown, additional databases may be linked to the controller 200 in a known manner. The controller 200 may include a program memory 215, a processor 220 (may be called a microcontroller or a microprocessor) for executing computer executable instructions, a random-access memory (RAM) 225 for temporarily storing data related to the computer executable instructions, and an input/output (I/O) circuit 230 for accepting and communicating the computer executable instructions, data for producing results with the computer executable instructions that are executed on the processor 220, and the results of any executed computer executable instructions. In one embodiment, the program memory 215 includes a compliance module 232 to implement a method for directing a patient to complete an action to bring the patient into compliance with an assigned healthcare protocol, as described below in relation to FIG. 3. In another embodiment (not shown) the compliance module 231 may be a separately-implemented IC. Of course, many other implementations of the compliance module 231 are possible. The compliance module may also include a plurality of modules to implement the method, for example, a detection module 232, a communication module 233, and an implementation module 234. The compliance module 231, and the plurality of modules 232, 233, 234 are discussed below in relation to FIG. 3.

The program memory 215, processor 220, and RAM may be interconnected via an address/data bus 235. It should be appreciated that although only one processor 220 is shown, the controller 200 may include multiple processors 220. Similarly, the memory of the controller 200 may include multiple RAMs 225 and multiple program memories 215. Although the I/O circuit 230 is shown as a single block, the I/O circuit 230 may include a number of different types of I/O circuits. The RAM(s) 225 and program memories 215 may be implemented as semiconductor memories, magnetically readable memories, and/or optically readable memories, for example. The controller 200 may also be operatively connected to the network 115 (FIG. 1) via a link 235.

The methods illustrated in the figures and described below may be implemented as computer-executable instructions on a variety network computers 110, servers 120, other network devices using a variety of wired and wireless networks and connections, or within a compliance module 231. Further, any action associated with the blocks described below and illustrated in FIG. 3 may be performed in any order, or at any time during the method 300 execution. With reference to FIGS. 1, 3, and 4, an interactive method 300 for facilitating patient compliance during a course of treatment, treatment plan, or other protocol by allowing the patient to directly implement an action to bring him or her into compliance is discussed and described.

At block 302, a patient may be notified of his or her non-compliance with a treatment plan or medical protocol and gain access to a compliance interface 400. As used herein, a treatment plan or protocol may be a course of treatment, a pharmaceutical regimen, a physical therapy regimen, or any other set of rules, procedures, or steps to which the patient is expected or required to comply that may be assigned or prescribed by a healthcare professional or voluntarily assumed by a patient. In one embodiment, a patient receives an email message from an entity 105 that includes a hyperlink or other predefined linkage from the email message to a compliance interface 400. The email message may be created manually or automatically upon satisfaction of rules, conditions, or other logic within the compliance module 231. For example, a detection module 232 within the compliance module 231 may determine when a patient is not complying with a protocol or may detect an out-of-compliance event associated with a patient participating in a protocol. For example, a healthcare professional or an EHR system at an entity 105 may receive and evaluate test results, prescription medication information, or other data and determine that the patient is not complying with a protocol. The healthcare professional or EHR system may then send the email including the hyperlink to the patient though a communication module 233 of the compliance module 231. Also, the professional or EHR system may notify a compliance manager or other third party entity 105 of the non-compliance. Upon receiving the notification, the compliance manager or other third party entity 105 may compose and send the email to the patient. Of course, many other methods may notify a patient of his or her non-compliance to a medical protocol and allow the patient to gain access to the compliance interface 400.

In one embodiment, the hyperlink within the email message includes a Universal Resource Locator (URL) that, when selected by the patient at a network computer 110, communicates GETs and POSTs (or any other message, such as an FTP command or an SNMP message that may request and/or communicate parameters to the method 300) from or to an entity 105. A launching URL may include a plurality of parameters to pass information about the patient to an entity 105 or to the method 300. For example, the launching URL may include a partner ID, a plan ID, and employer ID, a patient ID, a compliance message ID, or a message occurrence.

The launching URL may also accommodate parameters specific to the compliance message ID (e.g., test results that are displayed within the compliance interface 400), a modification of the method 300, or a parameter to ignore the parameters. The parameters may be used by an entity 105 to instantiate an application, routine, or other computer-executable instructions represented by the method 300. For example, selecting a hyperlink from an email message may pass parameters that identify a patient ID and a compliance message ID for the compliance interface 400 (e.g., Member #1704, Compliance Message #17) to the method 300. The passed parameters may also include test results or other data that may be used by the method 300 and may be presented to the patient in the compliance interface 400, as explained below. The parameters may also be encoded so that any patient information is not visible or accessible by any entity 105 that has not been approved by the patient. Further, any communication or transferal of medical or personal information may be compliant with the Health Insurance Portability and Accountability Act (HIPAA), or other legal standards.

Also, the method 300 may terminate and generate an error message if one of the parameters is missing or malformed or, alternatively, may allow the method 300 to continue. In one embodiment, receipt of missing or malformed parameters at a network computer 110 may initiate a number of operations to resolve any of the damaged parameters or to provide any missing parameters. For example, the method 300 may initiate a login screen for a patient to enter membership or other information in order to supply or correct missing parameters. Alternatively, the message or other command may initially direct a patient to a login screen to enter security or other information to confirm the patient's identity before the method 300 presents any medical or other personal information. In a further embodiment, the URL identifies a patient and a compliance message, but does not contain any parameters. Of course, many other sources of email messages, combinations of parameters, and delivery methods are available to direct or re-direct a patient to the compliance interface 400.

A patient may also be notified of his or her non-compliance and gain access to the compliance interface 400 by logging into a network-implemented, healthcare portal or interface either voluntarily, at the request of an entity 105, or through an email message sent by an entity. The healthcare interface may include access to an EHR, as previously described, from an entity 105 such as a hospital, insurance provider, compliance manager, or other third party entity 105. In one embodiment, a hospital or other entity notifies the patient (via mail, email, telephone, or other form of communication) that a compliance message has been sent to the patient's EHR. The EHR may include an indication that the patient is not in compliance with a protocol. The indication may be selectable by the patient and, upon selection, the method 300 routes the patient to the compliance interface 400.

Upon routing the patient to the compliance interface 400, the method 300 may observe and/or record any number of events related to the patient's interaction with the method 300. For example, each block of the method 300 may include a plurality of events that may be observed or recorded to a database 205. The recorded events may be associated with each instance or “session” of the method 300. The events may include a start time of the method 300, a time the patient entered a block of the method 300, a time the patient exited a block of the method 300, when or if the patient asked a question or performed the action, text of a question, if asked, and an entity 105 to which the question was asked, and selected or entered reasons, if any, for quitting the method 300. Each of these events is described in further detail below. Further, the method 300 may record the events to a database 205. The database 205 may store data related to the method 300 each time the method 300 is executed. Further, the database 205 may associate collected data with a discrete method 300 session, wherein the data may be keyed on a particular iteration or instantiation of the method 300. The data stored in the database 205 may include the parameters, as previously discussed.

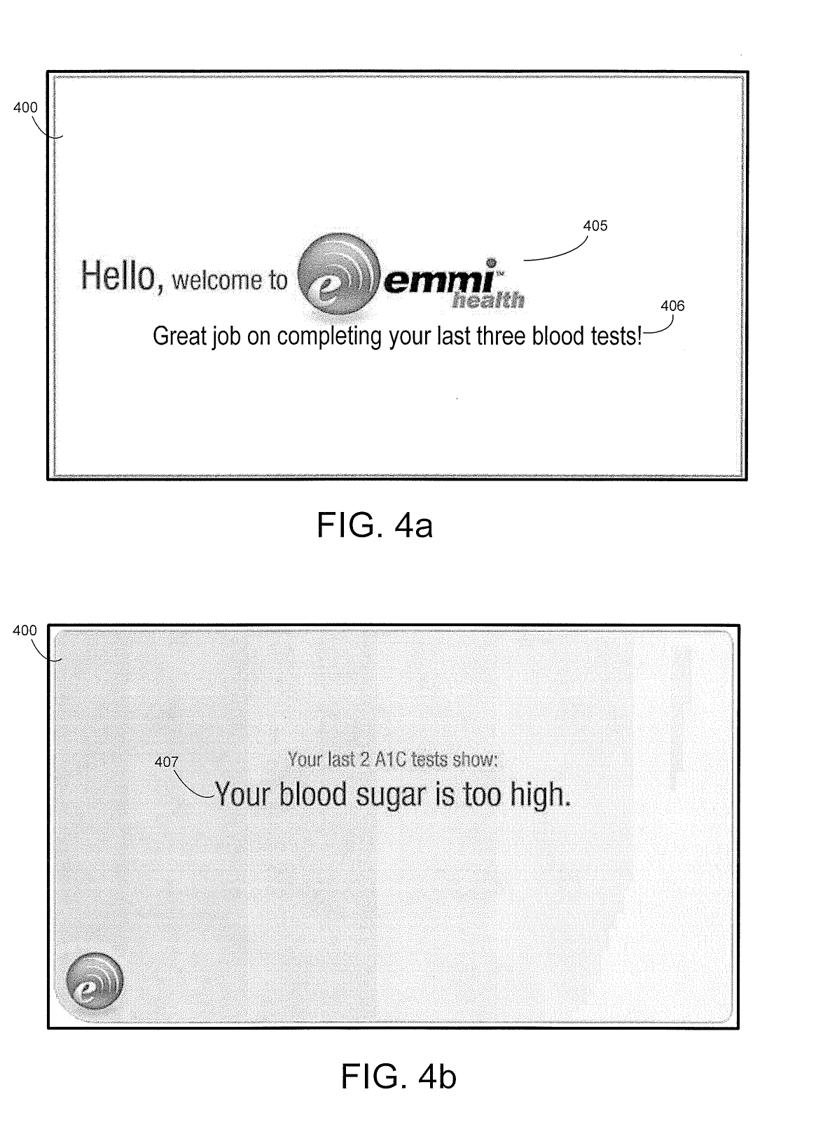

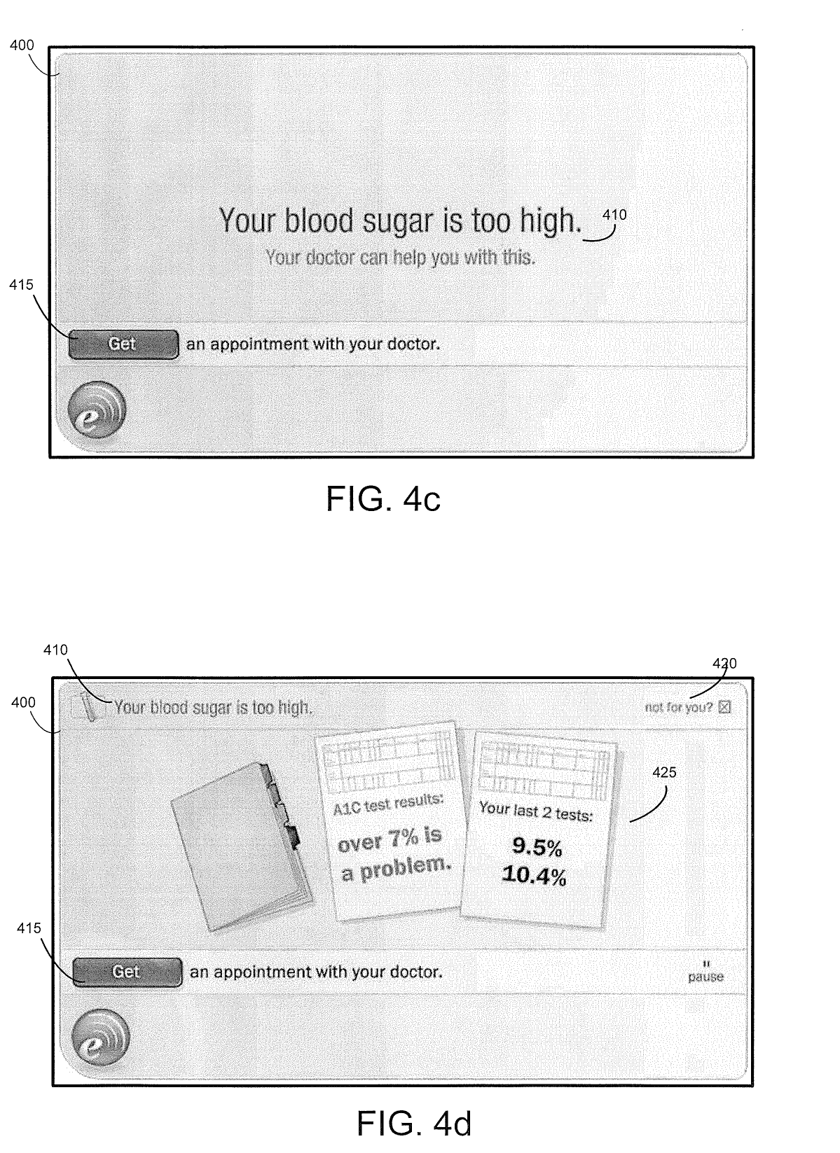

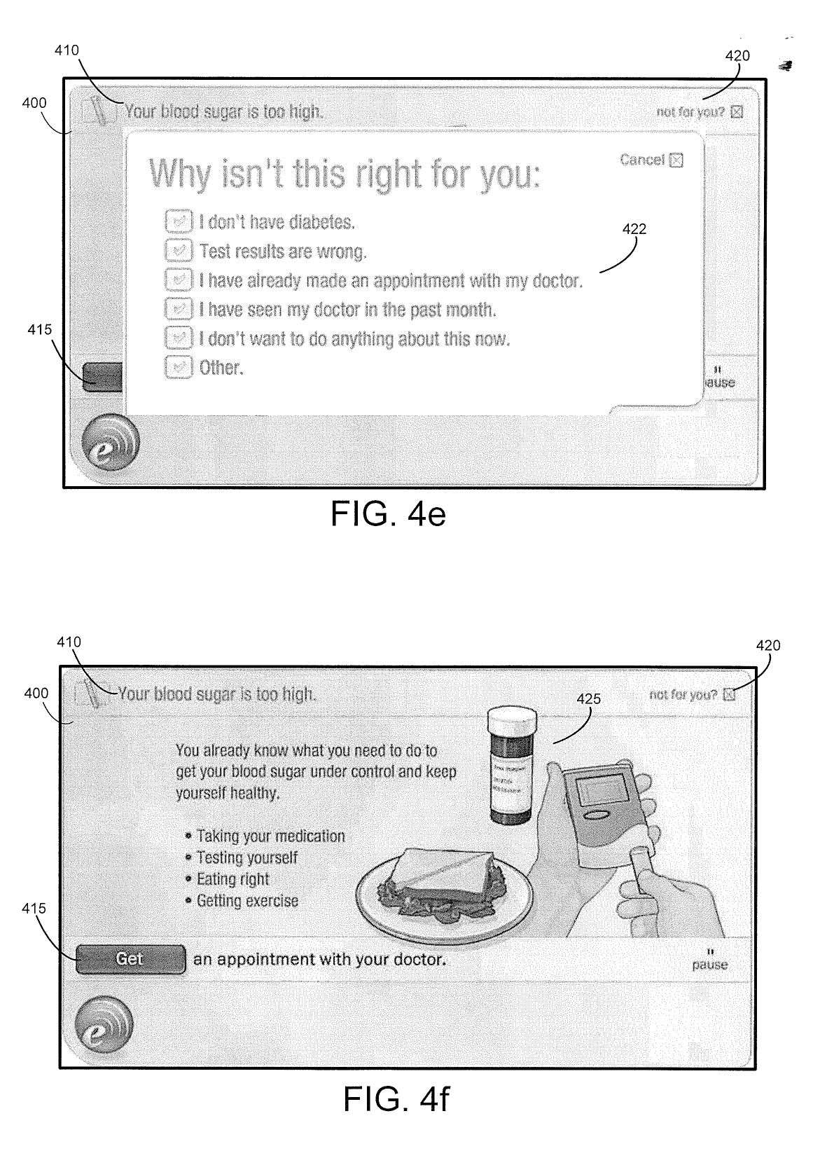

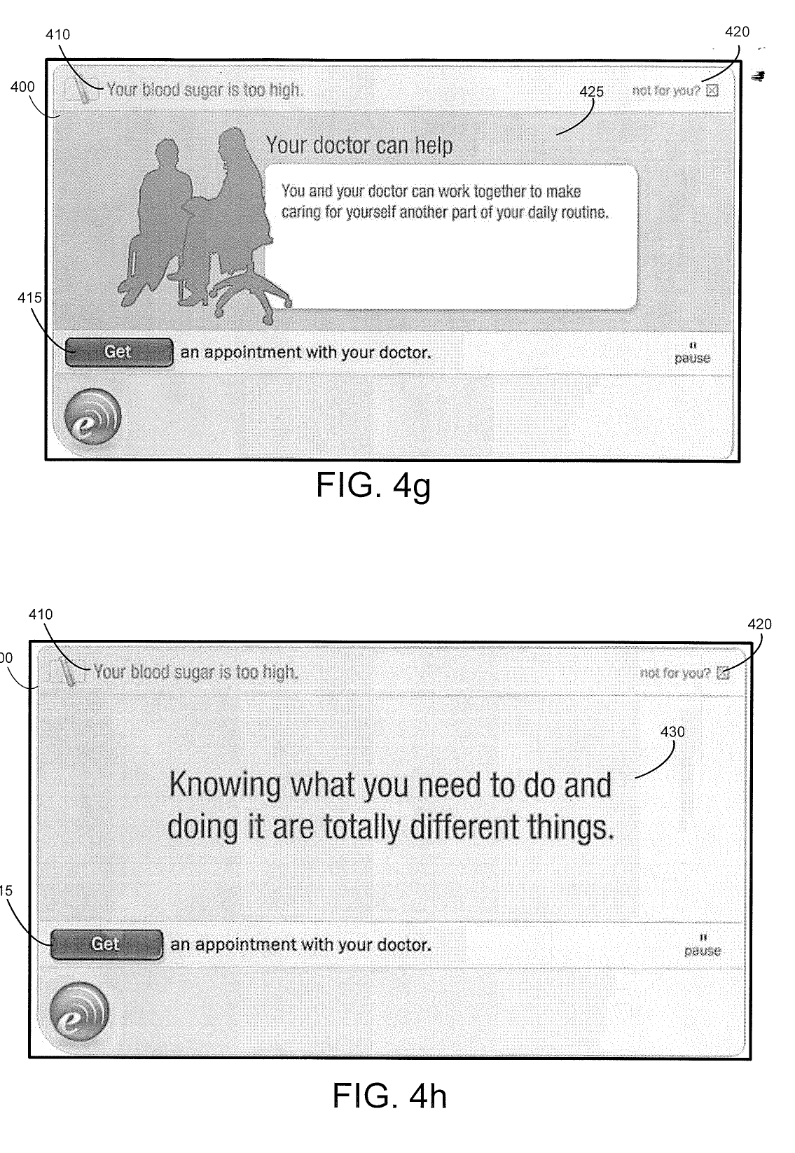

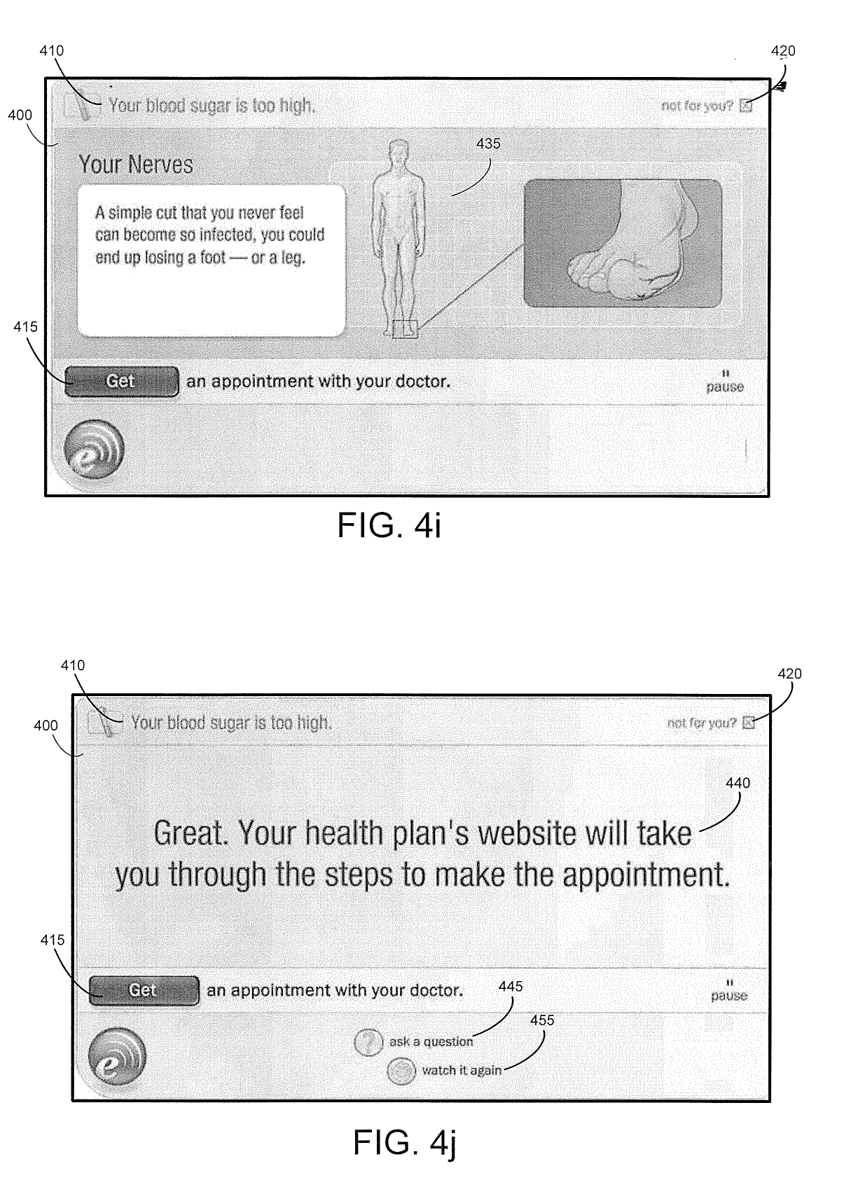

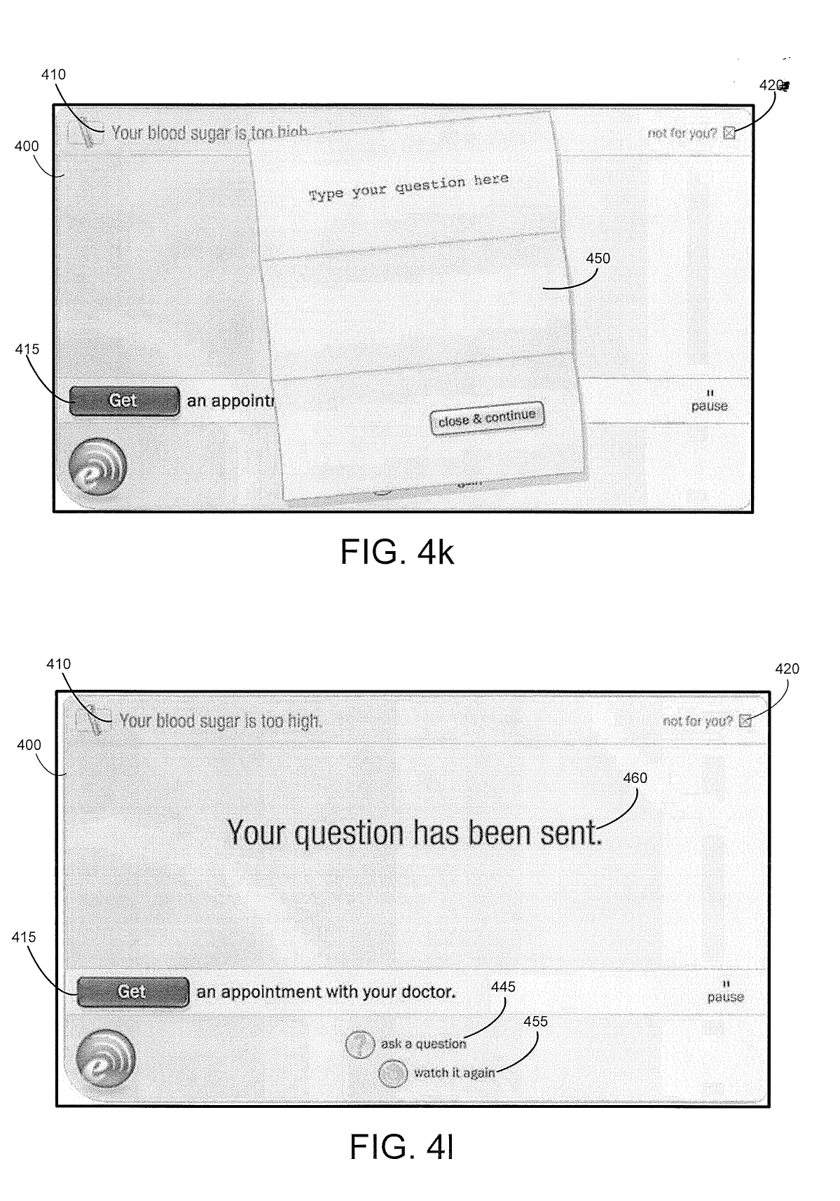

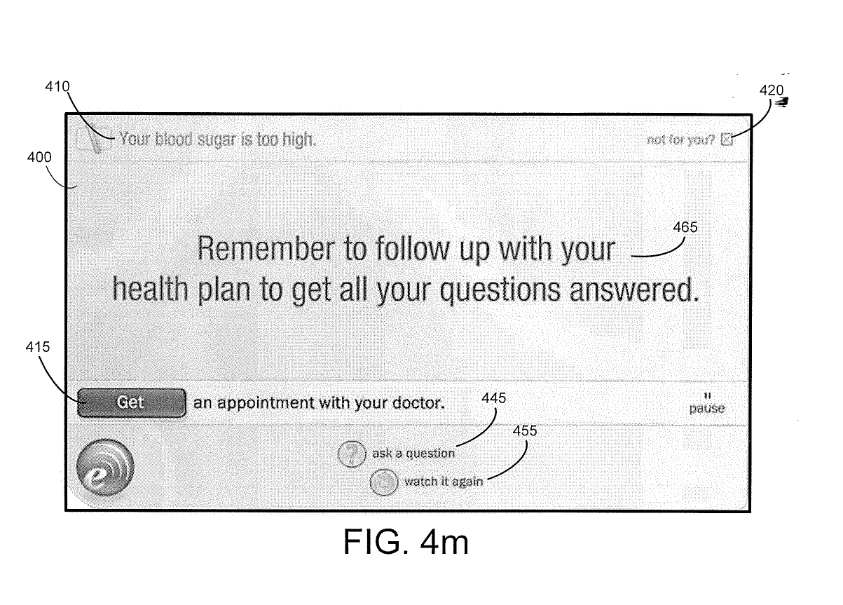

At block 305, a welcome message 405 (FIG. 4a) may be communicated from an entity 105 to a patient at a network computer 110. In one embodiment, the patient receives the welcome message at the patient's personal computer as a response from a server. The welcome message 405 may include a dynamic element 406 that indicates if the patient has previously reviewed this compliance message, has recently completed an action that brings the patient into compliance with a protocol, or any other information related to the patient or an action taken by the patient. For example, the dynamic element 406 may communicate “Great job on completing your last three blood tests!” or “Thank you for recently seeing your cardiologist for your follow-up appointment. You are on your way to a complete recovery!” or other information that is related to a patient's recent actions. The method 300 may also determine whether or not the patient has previously accessed or viewed information at the website and change the welcome message accordingly. In one embodiment, the method 300 accesses a number of previously recorded events, as described above, that are associated with the patient. For example, the method 300 may access the recorded events associated with the patient. If the method 300 determines that there are previously recorded events associated with the patient, the method 300 may change the dynamic element 406 of the welcome message to include a fact about the previous events. However, if the method 300 detects no previous events, the method 300 may determine that the patient has not previously viewed messages from the provider, and the dynamic element 406 of the welcome message may indicate that this is the patient's first visit, or may be absent from the interface 400. Further, a patient may have previously viewed information through the website and accomplished a task assigned during the previous visit as described below in relation to block 335. Upon logging in after completing the task, the welcome message may indicate that the patient completed the previous task or provide a similar greeting that may be specific to the previous visit. In a further embodiment, the method 300 may provide a unique welcome message based on the prior visit alone, provide the patient with a simple, unrelated greeting (e.g., “Hello!”), or communicate a welcome message that is specific to the entity 105 (e.g., “Thank you for accessing XYZ Health Services”).

The content presented to the patient within the compliance interface 400 may be modular and adaptable to a variety of interactive presentations. The content may be shared between a plurality of presentations, such as between a compliance message regarding diabetes and a compliance message regarding a patient's post-surgical protocol. Also, the content may include standard classes of information that correspond to specific message 410 and action 415 types (FIG. 4c), or the patient's health record information. For example, a standard class of information may be “test results.” A message 410 within the “test results” class may be modified to accommodate a patient's specific test results by instantiating the method 300 with patient data and patient test results. In response to abnormal test results, the detail message 425 may include a template of information that compares the patient's results with normal test results. The message 410 and action 415 may be tailored to a variety of other protocols. For example, if the patient has completed one of a plurality of sequential actions related to a medical device, a template may present information tailored to medical devices. Also, if the patient has completed or will complete a surgical procedure, a template may present information customized for surgical procedures.

Further, the method 300 may present the information within the compliance interface 400 in a standardized way. For example, information may be presented in a fixed arrangement of text, audio, or video for the test results, medical devices, or surgical procedure scenarios described above. Audio, video, and text content may also be accessed from disparate sources to be presented to the patient. The location of the information may be explicit, or from a common directory to be shared among many instantiations of the method 300 that are associated with related subjects.

At block 310, a compliance message 407 (FIG. 4b) may be communicated to the patient. The compliance message may include both a message 410 (FIG. 4c) and a selectable action 415. In one embodiment, either or both of the message 410 and the selectable action 415 may be retrieved directly from the patient's EHR at an entity 105. In another embodiment, the message 410 is sent by the entity 105 to a third party to be distributed to the patient via email or other method, for example, through the communication module 233. Upon communication of the compliance message 407, the patient may be able to take the action as described below in relation to block 335. The message 410 may present a variety of information to the patient regarding his or her health. For example, the message 410 may present parameters that are passed to the method 300 (as described above in relation to block 302), test results, a diagnosis based on the test results, a recommendation from the patient's health care provider (e.g., the patient should schedule a follow-up appointment, the patient should schedule a repeat of a previous test, the patient should be screened for another disease, etc.), advice or actions related to a medical device, messages generated to coincide with the progression of a disease, or any other message related to the patient's health care that may require further action.

The selectable action 415 may allow a patient to directly implement an act to influence his or her healthcare or may provide a patient with the resources to implement a healthcare act (e.g., schedule an appointment, schedule a test, obtain more information, submit a prescription, contact an entity 105, etc.). In one embodiment, an implementation module 234 of the compliance module 231 directly implements one or more steps of a healthcare protocol to bring the patient into compliance. The action 415 may further include any act that is related to the message 410 that may resolve a complication indicated by the message 410, may bring a patient into compliance with a course of treatment or other protocol, or may improve a patient's adherence to the protocol. For example, if the message 410 indicates a diagnosis or other conclusion based on test results that may require further attention, the action 415 may allow the patient to schedule an appointment with his or her health care provider. In one embodiment, the method 300 may be interfaced with an appointment scheduling system of an entity 105 to facilitate the action 415.

The action 415 may also permit the patient to obtain further information about the message 410 including allowing the patient to research information related to the message 410 from an external source such as the Internet, or from an internal source such as an on-line library stored on a network computer 110. The action 415 may also direct the patient to other sources of information, may allow the patient to submit further information, or may permit the patient to repeat an action that precipitated the initial compliance message 407. For example, the patient may have personal information that, if known, would explain the information contained in the compliance message 407. One situation may be that the patient consumed a type of food or a medication that he or she knew would likely result in inaccurate test results. In this situation, the action 415 may permit the patient to submit the personal information to a healthcare professional or an administrator that may evaluate the information to determine if another test is necessary. The administrator may then modify the method 300 to permit the patient to re-schedule the test or may provide additional information to the patient to avoid making the same mistake.

In one embodiment, the patient may select the action 415 as soon as it is displayed. In a further embodiment, the patient may only complete the action after viewing messages 410 or other information related to the patient's health. Further, the patient may be presented with a plurality of actions 415 to take based on the presentation of messages 410. In a still further embodiment, the action 415 sends the patient to another web-based presentation to complete an informed consent presentation related to a surgical procedure or a consent to release medical data. For example, the method 300 may interface with other interactive healthcare applications, programs, or libraries such as Emmi® Success™, Emmi® Prep™ Emmi® Health™, or Emmi® Kids™ produced by Emmi Solutions, LLC of Chicago, Ill.

The message 410 and action 415 may be presented in a variety of media formats. For example, the message 410 and action 415 may be presented to the patient in any combination of text, audio, or video that is delivered in any format. In one embodiment, the message 410 remains visible to the patient throughout the method 300. Further, the action 415 may include any object or web-based structure that is recognizable to the patient as being selectable. For example, the action 415 may take the form of a button, an input field, or a slide bar. In one embodiment, a button may consist of a standard button and a graphic overlay specific to the method 300.

The patient may also be presented with a selectable quitting option 420 (FIG. 4d). The quitting option 420 may allow the patient to stop the message and close the application or otherwise end the method 300. The quitting option 420 may be visible to the patient for all or substantially all of the method 300. Selecting the quitting option 420 may also present a number of alternatives to the action 415 that may be more desirable to the patient. The alternatives may be linked to data in the patient's health record that may indicate acceptable, though less-desirable alternatives to the presented action 415. Further, upon selection of the quitting option 420, the patient may, at block 312, be asked to choose a reason 422 (FIG. 4e) that the message 410 is not appropriate for them. In one embodiment, where the compliance message is related to diabetes protocol, upon selecting the quitting option 420, the method 300 displays reasons 422 for quitting including “I don't have diabetes,” “Test results are wrong,” “I have already made an appointment with my doctor,” “I have seen my doctor in the past month,” “I don't want to do anything about this now,” and “Other.” The reasons 422 may be accessed from a variety of sources. In one embodiment, the reasons 422 are pulled from a script associated with the method 300. Additionally, the reasons 422 may be pulled from a server 120 or a database 205.

Several events may occur upon selecting one or more of the reasons 422. In one embodiment, the selected reason 422 and any data entered by the patient is passed to an entity 105, such as a health care plan administrator. In a further embodiment, upon selecting any of the reasons 422, more compliance information or another action 415 may be displayed to the patient. For example, upon selecting an “I don't have (condition)” reason, the method 300 may present an action 415 that allows the patient to explore possible reasons for the test results leading to the condition, to submit an error message to an administrator, to call an administrator, or to review the patient's EHR to resolve the mistake. Selecting the “Test results are wrong” reason 422 may allow the patient to re-schedule a test, submit correct test data, or present reasons that the current test data may be incorrect. Selecting the “I have already made an appointment with my doctor” or the “I have seen my doctor in the past month” reason 422 may allow the patient to enter a date of the appointment, a confirmation code to update the system, or call or write an administrator. Selecting the “I don't want to do anything about this now” may cause the method 300 to terminate or may present additional reasons that the patient should complete the suggested action. For example, the method 300 may present information to encourage the patient to comply with the protocol, such as a worst-case scenario, or an escalating reason that the patient should comply. Selecting the “Other” reason 422 may present the patient with an editable text field in which to type a reason for quitting. In a still further embodiment, a patient's responses to the reasons 422 may be accumulated and passed to an entity 105 for further analysis. For example, statistical analysis of patients' reasons for non-compliance may allow an entity 105 to modify its practices to increase compliance. Also, the window displaying the message 410 and action 415 may close and the patient may be directed to another web page or, at block 313, directed to the provider, or the method 300 may terminate. The patient may also have the option of canceling out of the reasons 422. If the patient cancels the reasons 422 after selecting the selectable quitting option 420, the presentation may resume at the point at which the patient originally selected the option.

At block 315, the method 300 may present the patient with detail 425 (FIG. 4d) regarding the message 410 and the action 415. The detail 425 may include a reasoning for the message 410 and resulting action 415. The detail 425 may also be conditioned by other information, such as the patient's health record information or other parameters that may have been passed to the entity 105, as previously discussed. For example, the detail 425 may be presented to the patient by comparing information from the patient's health record to other information such as test data or statistical information to allow the patient to fully comprehend the justification for the action 415. Also, the detail 425 may include information related to the patient's condition, disease, device, or surgical procedure. For example, in FIGS. 4f and 4g, the detail 425 information includes information that is specific to the patient's current diagnosis (e.g., information to help the patient reduce his or her blood sugar level) and information to keep the patient in compliance with the protocol.

At block 320, the patient may be presented with a motivational message 430 (FIG. 4h) that links the detail 425 with a subsequent message. The motivational message 430 may be derived from the patient's current condition, the message 410, or the action 415 as it relates to historic treatment data. In one embodiment, the patient may be presented with a “teaser” message that personalizes the relationship between the condition or diagnosis that prompted the compliance message 407 and the detail 425. For example, the message 425 may include typical reasons patients have historically not performed the action 415 in response to the message 410.

At block 325, the patient may receive additional information as one or more reasons to take action 435 (FIG. 4i). A reason to take action 435 may include more detailed information about the patient's message 410 and action 415. Also, the reason to take action may explain a specific risk or benefit associated with the patient's diagnosis, disease, or device. In one embodiment, the patient may receive information explaining a consequence of not taking action.

At block 330, the patient may receive a conclusion message. In one embodiment, the conclusion message may allow the patient to view the previously-presented information In a further embodiment, the conclusion may allow the patient to view additional information regarding the message 410 or action 415. The conclusion may also redirect a patient to another website containing related health information that may be related to data contained within the patient's health record. The redirect website may also contain health information that is unrelated to the message 410 or the patient's record. Additionally, the conclusion message may allow the patient to review the message 410 and detail 425 as described in relation to blocks 310-325 and to, optionally, select the action 415.

In connection with any of the previously-described blocks, and at block 335, the patient may select the action 415. Upon selection, the patient may complete or schedule an event related to the message 410 that brings the patient into compliance with a protocol, as previously described. The redirection may be partner, plan, employer, and message 410 specific. In one embodiment, the event corresponding to the action 415 is dependent on one or more factors associated with the patient who is viewing the message 410. For example, the action may be dependent on the patient type and the healthcare plan in which the patient is enrolled. If the patient is a member of a healthcare benefits plan that permits scheduling appointments, then, at block 313, selecting the action 415 may re-direct the patient to a website associated with the healthcare benefits plan to schedule an appointment. If the patient receives his benefits through an employer that also maintains work site healthcare facilities for the primary policy holder, then selecting the action 415 may direct the primary policy holder to a website to schedule an appointment at the work site facilities. If the same policy provides insurance coverage for spouses, but not for the work site facilities, then selecting the action 415 by a spouse my re-route the spouse to a different scheduling website to complete the action 415. Of course, many factors other than patient type and healthcare plan may determine the result of selecting the action 415. The patient may also be presented with an acknowledgement message 440 (FIG. 4j) that informs the patient of the next steps involved in performing or completing the action 415. The patient may also be presented with an option to ask a question 445 and an option to view the information again 455.

At block 345, the patient may select the option to ask a question 445 and optionally ask a question 445 regarding any of the information that the method 300 has previously presented. In one embodiment, the method 300 may not present the patient with the option of asking a question until after a first presentation of a conclusion message, as described above in relation to block 330. Upon selecting the option to view the information again 455 (described below in relation to block 350), the patient may be presented with the option 455 throughout the interactive presentation. In a further embodiment, the patient may be presented with a virtual form 450 (FIG. 4k) on which he or she may type a question to submit to another entity. For example, the patient may be able to type a question on the form 450 and, at block 350, submit it to his or her health care provider. Additionally, the patient may send the question to a number of different health care providers in search of a second opinion. Any or all of the patient's health record data may be sent along with the question. In a still further embodiment, only the patient's health record information pertaining to the current message 410 or action 415 may be sent. When a question is sent to a health care provider to which the patient has not previously given consent for release of personal health record data, the patient may also be asked to execute an online consent form or otherwise provide consent to the outside provider before the data may be sent. After sending the question, the patient may be presented with an acknowledgement that the message has been sent 460 (FIG. 4l). In a still further embodiment, after submitting the question, the method 300 resumes the presentation at the point at which the patient began the question process. In a still further embodiment, the provider may optionally enable or disable the patient's ability to ask questions.

At block 350, the patient may select the option to view the information again 455 and optionally begin the presentation again. In one embodiment, selecting the option 455 directs the patient to the motivational message 430 as previously described in relation to block 320. Of course, any other portion of the presentation may be presented to the patient upon selection of the option. Further, at any time after viewing the motivational message, the patient may select the quitting option 420, as previously described in relation to block 312. Upon one or more of the patient quitting the presentation (block 312) or the patient taking the action (block 335), the patient may receive a closing statement 465 (FIG. 4m) that indicates the method 300 has terminated. In one embodiment, at termination 355, the patient may be redirected to another website.

Data may also be passed to another entity based on the patient's actions during the method 300. For example, as previously described, the patient's reason for quitting the method 300, as well as selecting an action 415, or submitting a question may be transferred to the provider or any other entity. The data may be passed either synchronously as the action occurs, or asynchronously as a cumulative data dump to one or more entities. The data may be passed over secure FTP, and may be in the form of a spreadsheet, text file, proprietary data file, or other structure and may be encrypted.

Much of the inventive functionality and many of the inventive principles are best implemented with or in software programs or instructions and integrated circuits (ICs) such as application specific ICs. It is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation. Therefore, in the interest of brevity and minimization of any risk of obscuring the principles and concepts in accordance to the present invention, further discussion of such software and ICs, if any, will be limited to the essentials with respect to the principles and concepts of the preferred embodiments.

Although the forgoing text sets forth a detailed description of numerous different embodiments, it should be understood that the scope of the patent is defined by the words of the claims set forth at the end of this patent. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

Thus, many modifications and variations may be made in the techniques and structures described and illustrated herein without departing from the spirit and scope of the present claims. Accordingly, it should be understood that the methods and apparatus described herein are illustrative only and are not limiting upon the scope of the claims.

What is claimed:

1. A method for facilitating a protocol comprising:
communicating a compliance message to a patient if the patient is not in compliance with one or more steps of the protocol, the compliance message including a message and a selectable action;
communicating the message to the patient, the message including healthcare protocol information indicating that the patient is not in compliance with the protocol; and
directing the patient to a resource if the patient selects the selectable action;
wherein the resource allows the patient to complete the one or more steps of the protocol to bring the patient into compliance with the one or more steps of the protocol;
wherein the compliance message is communicated to the patient if the one or more steps of the protocol is not completed.

2. The method of claim 1, wherein the one or more steps of the healthcare protocol are assigned to the patient by a healthcare professional.

3. The method of claim 1, wherein the patient voluntarily assumes the one or more steps of the protocol.

4. The method of claim 1, wherein the protocol includes one or more of a course of treatment, a pharmaceutical regimen, a physical therapy regimen, a set of rules, or a plurality procedures.

5. The method of claim 1, further comprising one or more of detecting if the patient is not in compliance with the protocol and detecting if the patient has completed the one or more steps of the protocol.

6. The method of claim 1, wherein communicating the compliance message to the patient if the patient is not in compliance with the one or more steps of the protocol comprises communicating the compliance message to the patient upon detecting that the patient has not completed the one or more steps of the protocol.

7. The method of claim 1, wherein communicating the compliance message to the patient if the patient is not in compliance with the one or more steps of the protocol comprises communicating an email message to the patient, the email message including a hyperlink.

8. The method of claim 7, further comprising redirecting the patient from a compliance interface to the resource using the hyperlink.

9. The method of claim 1, further comprising communicating the compliance message to an electronic health records system assigned to the patient.

10. The method of claim 1, wherein the message includes one or more of text, audio, or video.

11. The method of claim 1, further comprising accessing a compliance interface including the compliance message.

12. The method of claim 11, wherein accessing the compliance interface includes communicating to the compliance interface, one or more parameters including one or more of a partner ID, a plan ID, an employer ID, a patient ID, a compliance message ID, a message occurrence, and a patient test results.

13. The method of claim 12, further comprising modifying one or more of the message and the selectable action with the one or more parameters.

14. The method of claim 1, further comprising modifying one or more of the message and the selectable action if the compliance message has been previously communicated to the patient.

15. The method of claim 1, wherein the compliance message includes a welcome message, the welcome message including a dynamic element.

16. The method of claim 15, further comprising modifying the dynamic element upon determining one or more of:
the patient selecting the selectable action,
a previous communication of the compliance message to the patient,
that the patient is not in compliance with the protocol,
that the patient has completed a step of the protocol, and
that the patient is in compliance with a different protocol.

17. The method of claim 1, wherein communicating the compliance message to the patient comprises communicating the compliance message to the patient within a compliance interface.

18. The method of claim 1, wherein the message includes one or more of test results, a diagnosis based on the test results, a recommendation from a healthcare provider, a recommendation that the patient should schedule a follow-up appointment, a recommendation that the patient should schedule a repeat of a previous test, a recommendation that the patient should be screened for another disease, a recommendation related to a medical device, a message generated to coincide with a progression of a disease, and a message requiring further action that is related to the protocol.

19. A computer readable medium including computer executable instructions to implement a method to direct a patient to complete a selectable course of action to bring the patient into compliance with a healthcare protocol that is assigned to the patient by an entity, the method comprising:
detecting an out-of-compliance event for the patient participating in the healthcare protocol, wherein the out-of-compliance event indicates that the patient is not in compliance with one or more procedures of the healthcare protocol;
communicating a compliance message from the entity to the patient, the compliance message including a message and the selectable course of action, the message including a reason that the patient is not in compliance with the one or more procedures of the healthcare protocol, and the selectable course of action allowing the patient to comply with the one or more procedures of the healthcare protocol; and
allowing the patient to directly implement the one or more procedures of the healthcare protocol upon selection of the selectable course of action;
wherein the one or more procedures of the healthcare protocol includes one or more of scheduling an appointment, scheduling a test, obtaining more information, submitting a prescription, repeating a healthcare action that precipitated the compliance message and, contacting the entity.

20. The computer readable medium of claim 19, further comprising redirecting the patient to an appointment scheduling system of the entity upon selection of the selectable course of action.

21. The computer readable medium of claim 19, further comprising:
communicating patient compliance information to the entity, the patient compliance information indicating that the patient is in compliance with the one or more procedures of the healthcare protocol;
modifying the compliance message upon receipt of the patient compliance information;
wherein modifying the compliance message upon receipt of the patient compliance information comprises one or more of permitting the patient to re-schedule the test, and providing additional information to the patient to avoid future non-compliance.

22. The computer readable medium of claim 19, further comprising communicating, to the patient, one or more alternatives to the one or more procedures of the healthcare protocol.

23. The computer readable medium of claim 19, further comprising allowing the patient to quit the compliance message, and, upon the patient quitting the compliance message, one or more of:
communicating, to the patient, one or more alternatives to the one or more procedures of the healthcare protocol;
communicating, to the entity, one or more reasons for quitting the compliance message; and
submitting an error message to an administrator.

24. The computer readable medium of claim 19, further comprising communicating an escalated message to the patient upon one or more of:
the patient selecting the selectable course of action after an extended period of time, and
the patient quitting the compliance message,
wherein the escalated message to the patient includes additional benefits of the patient completing the one or more procedures of the healthcare protocol.

25. A computer system with a compliance module for directing a patient to complete an action to bring the patient into compliance with an assigned healthcare protocol comprising:
a first network computer including a first processor;
a data network operatively coupled to the first network computer;
the compliance module coupled to the data network and operatively coupled to a second processor and a memory storing computer-executable instructions for executing a program, the program comprising:
a detection module for detecting an out-of-compliance event for the patient participating in the assigned healthcare protocol, wherein the out-of-compliance event indicates that the patient is not in compliance with one or more procedures of the assigned healthcare protocol;
a communication module for communicating a compliance message through the data network to the patient at the first network computer, the compliance message including a message and a selectable course of action, the message including a reason that the patient is not incompliance with the assigned healthcare protocol, and the selectable course of action allowing the patient to comply with the one or more steps of the assigned healthcare protocol; and
an implementation module for allowing the patient to implement the one or more steps of the assigned healthcare protocol upon selection of the selectable course of action.
